(19) United States
(12) Patent Application Publication
(10) Pub. No.: US 2021/0024966 A1
(43) Pub. Date: Jan. 28, 2021

(54) METHOD OF OBTAINING A MICROBIAL OIL AND A METHOD OF REDUCING EMULSION BY MAINTAINING A LOW CONCENTRATION OF CARBOHYDRATE

(71) Applicants: DSM IP Assets B.V., HEERLEN (NL); Evonik Operations GmbH, Essen (DE)

(72) Inventors: Martin HEINING, Karlstein am Main (DE); Michael Benjamin JOHNSON, Columbia, MD (US); Neil Francis LEININGER, Omaha, NE (US); Shannon Elizabeth Ethier RESOP, Olney, MD (US); Justin Edward STANCZYK, Winchester, KY (US); Vinod TARWADE, Ellicott City, MD (US)

(21) Appl. No.: 17/042,788

(22) PCT Filed: Mar. 29, 2019
(86) PCT No.: PCT/US2019/024762

§ 371 Date: Sep. 28, 2020

Related U.S. Application Data
(60) Provisional application No. 62/650,354, filed on Mar. 30, 2018.
(60) Provisional application No. 62/652,602, filed on Apr. 4, 2018.

(51) Int. Cl. C12P 7/64

(52) U.S. Cl. CPC C12P 7/6427

(57) ABSTRACT

Disclosed herein are processes for reducing emulsion during the process of obtaining a microbial oil comprising one or more polyunsaturated fatty acids (PUFAs) from one or more microbial cells by maintaining the level of carbohydrate at less than 15 g/Kg in the fermentation broth. Further disclosed herein is microbial oil comprising one or more PUFAs that is recovered from microbial cells by at least one process described herein.

### CROSS REFERENCE TO RELATED APPLICATIONS

This application claims the benefit of the filing date of U.S. Provisional Patent Application Nos. 62/650,354 filed Mar. 30, 2018 and 62/652,602 filed Apr. 4, 2018, the disclosures of which are hereby incorporated by reference in their entirety.

### FIELD OF INVENTION

The present invention relates to a method of obtaining polyunsaturated fatty acids containing lipids from a lipid-containing biomass.

### BACKGROUND OF THE INVENTION

Disclosed herein are processes for obtaining a microbial oil comprising one or more polyunsaturated fatty acids (PUFAs) from one or more microbial cells. Further disclosed herein is a microbial oil comprising one or more PUFAs that is recovered from microbial cells by at least one process described herein.

Microbial oil containing one or more PUFAs is produced by microorganisms, such as, for example, algae and fungi.

A typical process for obtaining PUFA containing oil from microbial cells involves growing microorganisms that are capable of producing the desired oil in a fermenter, pond or bioreactor to produce a microbial cell biomass; separating the biomass from the fermentation medium in which the biomass was grown; drying the microbial cell biomass, using a water-immiscible organic solvent (e.g., hexane) to extract the oil from the dried cells; and removing the organic solvent (e.g., hexane) from the oil.

Another process for obtaining PUFA containing oil from microbial cells involves growing microorganisms that are capable of producing the desired oil in a fermenter, pond or bioreactor to produce a microbial cell biomass; releasing the PUFA containing oil into the fermentation medium in which the cells were grown by using mechanical force (e.g., homogenization), enzymatic treatment, or chemical treatment to disrupt the cell walls; and recovering the oil from the resulting composition comprising PUFA containing oil, cell debris, and liquid using a water miscible organic solvent. The oil can be separated mechanically from the composition and the alcohol must be removed from both the oil and the aqueous biomass waste stream.

More recently, a third, solvent-free method was developed for obtaining PUFA containing oil from microbial cells. The solvent-free process for obtaining PUFA containing oil from microbial cells involves growing microorganisms that are capable of producing the desired oil in a fermenter, pond or bioreactor to produce a microbial cell biomass; releasing the PUFA containing oil into the fermentation medium in which the cells were grown by using mechanical force (e.g., homogenization), enzymatic treatment, or chemical treatment to disrupt the cell walls; and recovering crude oil from the resulting composition comprising PUFA containing oil, cell debris, and liquid by raising the pH, adding a salt, heating, and/or agitating the resulting composition.

The above solvent-free process has the benefit of avoiding the use of a large amount of volatile and flammable organic solvent. This method, however, requires breaking of the thick emulsion that is generated after the cell is lysed and the oil is released and mixed with cell debris and fermentation broth components. This causes long oil recovery times, use of large amounts of salt, and/or many steps, which can all increase processing costs. In addition, the formation of emulsion during the cell lysing step reduces the efficiency of the oil extraction process and directly affects the extraction yield of such process.

As a result, there is a need for identifying the broth components that are responsible for the formation of emulsion and influencing oil quality, separation, and overall process efficiency. Success in identifying such components may lead to the reduction or even elimination of emulsion, thereby minimizing the number of steps in oil extraction, shorten oil recovery times, and help to provide a high yield of top quality PUFA containing oil.

### BRIEF SUMMARY OF THE INVENTION

The present invention is directed to a process for obtaining a microbial oil comprising one or more polyunsaturated acids from one or more microbial cells contained in a fermentation broth, wherein less than 15 g/Kg of carbohydrate is maintained in the fermentation broth during the process.

In one embodiment, the process further comprises:
(a) lysing the cells comprising the microbial oil to form a lysed cell composition;
(b) demulsifying the lysed cell composition to form a demulsified lysed cell composition;
(c) separating the oil from the demulsified lysed cell composition; and
(d) recovering the oil.

The present invention is also directed to a process for reducing the amount of caustic agent used in extracting a microbial oil comprising one or more polyunsaturated acids from one or more microbial cells contained in a fermentation broth, wherein less than 15 g/Kg of carbohydrate is maintained in the fermentation broth during the oil extraction process. In one embodiment, less than 18 g of caustic soda is used per 1 Kg fermentation broth.

In some embodiments, 0-10 g/Kg of carbohydrate is maintained in the fermentation broth during the above processes. In one embodiment, this level of carbohydrate is maintained in the fermentation broth before step (a).

In one embodiment, the microbial cells used above are capable of producing at least about 10 wt. %, at least about 20 wt. %, preferably at least about 30 wt. %, more preferably at least about 40 wt. % of their biomass as lipids. In some embodiments, the polyunsaturated lipids comprise one or any combination of DHA, EPA, and ARA.

In one embodiment, the carbohydrate used in the above process is select from glucose, sucrose, dextrose, polysaccharide, and mixtures thereof.

In one embodiment, the microbial cells are selected from algae, fungi, protists, bacteria, microalgae, and mixtures thereof. In the embodiment, the microbial cells are from the genus Mortierella, genus Crypthecodinium, or order Thraustochytriales. In another embodiment, the microbial cells are from the order Thraustochytriales. In another embodiment, the microbial cells are from the genus Thraustochytrium, Schizochytrium, or mixtures thereof. In yet another embodiment, the microbial cells are from Mortierella Alpina.

### BRIEF SUMMARY OF DRAWINGS

FIG. 1 is a diagram illustrating the experimental design to examine the influence of glucose on emulsion formation/phase separation during downstream process (DSP).

FIG. 2 shows the effect of varying amounts of glucose on emulsion when the glucose is added before pasteurization. b1: 0.2 g/Kg glucose (control), b2: 20 g/Kg glucose, b3: 40 g/Kg glucose, b4: 60 g/Kg glucose.

FIG. 3 shows the effect of addition of 20 g/Kg glucose on emulsion when the glucose is added at different stages of the DSP process. b1: 0.2 g/Kg glucose (control), b2: 20 g/Kg glucose added before pasteurization, b5: 20 g/Kg glucose added after pasteurization, b6: 20 g/Kg glucose added after cell lysis, b7: 20 g/Kg glucose added after broth concentration.

FIG. 4 shows the dependence of the amount of caustic required for breaking emulsion with different amount of residual glucose in the starting broth.

### DETAILED DESCRIPTION OF THE INVENTION

The features and advantages of the invention may be more readily understood by those of ordinary skill in the art upon reading the following detailed description. It is to be appreciated that certain features of the invention that are, for clarity reasons, described above and below in the context of separate embodiments, may also be combined so as to form sub-combinations thereof.

Embodiments identified herein as exemplary are intended to be illustrative and not limiting.

Disclosed herein is a process for obtaining a microbial oil comprising one or more polyunsaturated acids from one or more microbial cells, wherein the process comprises:

(a) lysing the cells comprising the microbial oil to form a lysed cell composition;
(b) demulsifying the lysed cell composition to form a demulsified lysed cell composition;
(c) separating the oil from the demulsified lysed cell composition; and
(d) recovering the oil;
wherein less than 15 g/Kg of carbohydrate is maintained in the cell composition during the process.

A particular advantage of the process described in the present invention is that the formation of emulsion is significantly reduced by maintaining a low or minimal amount of carbohydrates during the process. It was very surprising, according to the present invention, to find out that higher concentration of carbohydrate in the broth composition affects free oil separation efficiency. It was further found that when the amount of carbohydrate is reduced to a lower level, the formation of emulsion is reduced when comparing to a similar process where the level of carbohydrate is uncontrolled or is maintained at a higher level.

The preferred carbohydrate level has been identified in the present invention. In one embodiment, the concentration of carbohydrate in the fermentation broth is maintained at less than 15 g/Kg during the oil extraction process. In another embodiment, the concentration of carbohydrate in the fermentation broth is maintained at less than 14 g/Kg, less than 13 g/Kg, less than 12 g/Kg, less than 11 g/Kg, less than 10 g/Kg, less than 9 g/Kg, less than 8 g/Kg, less than 7 g/Kg, less than 6 g/Kg, less than 5 g/Kg, less than 4 g/Kg, less than 3 g/Kg, less than 2 g/Kg, less than 1 g/Kg, or less than 0.2 g/Kg. In another embodiment, the concentration of carbohydrate in the fermentation broth is maintained at between 5-10 g/Kg. In another embodiment, the concentration of carbohydrate in the fermentation broth is maintained at between 0.2-5 g/Kg. In another embodiment, the concentration of carbohydrate in the fermentation broth is maintained at between 5-15 g/Kg. In yet another embodiment, the concentration of carbohydrate in the fermentation broth is maintained at between 0-15 g/Kg. In yet another embodiment, the concentration of carbohydrate in the fermentation broth is maintained at between 0.2-15 g/Kg.

The role of sugars in emulsion formation was examined by addition of glucose at different stages of the extraction process. It was found that the amount of glucose added before pasteurization, which would be analogous to residual sugar in the fermentation broth, was mainly responsible for the emulsion formed during the extraction process.

Thus, in one embodiment, the concentration of carbohydrate in the fermentation broth is maintained at the end of the fermentation process but before the start of the oil extraction process, at less than 14 g/Kg, less than 13 g/Kg, less than 12 g/Kg, less than 11 g/Kg, less than 10 g/Kg, less than 9 g/Kg, less than 8 g/Kg, less than 7 g/Kg, less than 6 g/Kg, less than 5 g/Kg, less than 4 g/Kg, less than 3 g/Kg, less than 2 g/Kg, less than 1 g/Kg, or less than 0.2 g/Kg. in another embodiment, the concentration of carbohydrate in the fermentation broth is maintained at end of the fermentation process and throughout of the oil extraction process

The term “carbohydrate” refers generally to the carbon energy sources that is normally supplied in any fermentation broth. The carbohydrates which are commonly included in a fermentation broth include, but are not limited to, glucose, sucrose, dextrose and polysaccharide.

In one embodiment, the concentration of carbohydrate is set to less 15 g/Kg by exhausting the carbohydrate source at the end of the fermentation process. This may be achieved by, for example, running the fermentation process for a sufficient long period of time in order to let all or almost all the carbohydrate consumed by the cell in the fermenter. In another embodiment, excessive carbohydrate may be removed before the process of oil extraction in order to reduce the concentration of carbohydrate to less 15 g/Kg.

Another advantage of the process described in the present invention is that the amount of caustic soda used in the demulsification process is significantly reduced by maintaining a low or minimal amount of carbohydrates during the process. It is surprising to find out that higher concentration of carbohydrate in the lysed cell composition causes high amount of caustic soda usage to break emulsion.

The minimal level of caustic soda used has been identified in the present invention. In one embodiment, when the concentration of carbohydrate in the fermentation broth is maintained at less than 15 g per Kg of fermentation broth during the oil extraction process, less than 18 g/KG caustic soda may be used. In another embodiment, the concentration of carbohydrate in the fermentation broth is maintained at less than 14 g/Kg, less than 13 g/Kg, less than 12 g/Kg, less than 11 g/Kg, less than 10 g/Kg, less than 9 g/Kg, less than 8 g/Kg, less than 7 g/Kg, less than 6 g/Kg, less than 5 g/Kg, less than 4 g/Kg, less than 3 g/Kg, less than 2 g/Kg, less than 1 g/Kg, or less than 0.2 g/Kg. In another embodiment, the concentration of carbohydrate in the fermentation broth is maintained at between 5-10 g/Kg. In another embodiment, the concentration of carbohydrate in the fermentation broth is maintained at between 0.2-5 g/Kg. In another embodiment, the concentration of carbohydrate in the fermentation broth is maintained at between 5-15 g/Kg. In yet another embodiment, the concentration of carbohydrate in the fermentation broth is maintained at between 0-15 g/Kg. In yet another embodiment, the concentration of carbohydrate in the fermentation broth is maintained at between 0.2-15 g/Kg.

Also disclosed herein is a microbial oil obtained by any of the processes described herein.

The microbial oil described herein refers to oil that comprises one or more PUFAs and is obtained from microbial cells.

Polyunsaturated fatty acids (PUFAs) are classified based on the position of the first double bond from the methyl end of the fatty acid; omega-3 (n-3) fatty acids contain a first double bond at the third carbon, while omega-6 (n-6) fatty acids contain a first double bond at the sixth carbon. For example, docosahexaenoic acid (DHA) is an omega-3 long chain polyunsaturated fatty acid (LC-PUFA) with a chain length of 22 carbons and 6 double bonds, often designated as “22:6n-3.” In one embodiment, the PUFA is selected from an omega-3 fatty acid, an omega-6 fatty acid, and mixtures thereof. In another embodiment, the PUFA is selected from LC-PUFAs. In a still further embodiment, the PUFA is selected from docosahexaenoic acid (DHA), eicosapentaenoic acid (EPA), docosapentaenoic acid (DPA), arachidonic acid (ARA), gamma-linolenic acid (GLA), dihomo-gamma-linolenic acid (DGLA), stearidonic acid (SDA), and mixtures thereof. In another embodiment, the PUFA is selected from DHA, ARA, and mixtures thereof. In a further embodiment, the PUFA is DHA. In a further embodiment, the PUFA is EPA. In yet a further embodiment, the PUFA is ARA.

LC-PUFAs are fatty acids that contain at least 3 double bonds and have a chain length of 18 or more carbons or 20 or more carbons. LC-PUFAs of the omega-6 series include, but are not limited to, di-homo-gammalinoleic acid (C20:3n-6), arachidonic acid (C20:4n-6), docosatetraenoic acid or adrenic acid (C22:4n-6), and docosapentaenoic acid (C22:5n-6). The LC-PUFAs of the omega-3 series include, but are not limited to, eicosatrienoic acid (C20:3n-3), eicosatetraenoic acid (C20:4n-3), eicosapentaenoic acid (C20:5n-3), docosapentaenoic acid (C22:5n-3), and docosahexaenoic acid (C22:6n-3). The LC-PUFAs also include fatty acids with greater than 22 carbons and 4 or more double bonds including, but not limited to, C24:6(n-3) and C28:8(n-3).

The PUFAs can be in the form of a free fatty acid, salt, fatty acid ester (e.g. methyl or ethyl ester), monoacylglycerol (MAG), diacylglycerol (DAG), triacylglycerol (TAG), and/or phospholipid (PL).

Highly unsaturated fatty acids (HUFAs) are omega-3 and/or omega-6 polyunsaturated fatty acids that contain 4 or more unsaturated carbon-carbon bonds.

As used herein, a “cell” refers to an oil-containing biomaterial, such as biomaterial derived from oleaginous microorganisms. Oil produced by a microorganism or obtained from a microbial cell is referred to as “microbial oil”. Oil produced by algae and/or fungi is also referred to as algal and/or fungal oil, respectively.

As used herein, a “microbial cell” or “microorganism” refers to organisms such as algae, bacteria, fungi, yeast, protist, and combinations thereof, e.g., unicellular organisms. In some embodiments, a microbial cell is a eukaryotic cell. A microbial cell includes, but is not limited to, golden algae (e.g., microorganisms of the kingdom Stramenopiles); green algae; diatoms; dinoflagellates (e.g., microorganisms of the order Dinophyceae including members of the genus Crypthecodinium such as, for example, Crypthecodinium cohnii or C. cohnii); microalgae of the order Thraustochytriales; yeast (Ascomycetes or Basidiomycetes); and fungi of the genera Mucor, Mortierella, including but not limited to Mortierella alpina and Mortierella sect. schmuckeri, and Pythium, including but not limited to Pythium insidiosum.

In one embodiment, the microbial cells are from the genus Mortierella, genus Crypthecodinium, or order Thraustochytriales. In a still further embodiment, the microbial cells are from Crypthecodinium Cohnii. In yet an even further embodiment, the microbial cells are selected from Crypthecodinium Cohnii, Mortierella alpina, genus Thraustochytrium, genus Schizochytrium, and mixtures thereof.

In a still further embodiment, the microbial cells include, but are not limited to, microorganisms belonging to the genus Mortierella, genus Conidiobolus, genus Pythium, genus Phytophthora, genus Penicillium, genus Cladosporium, genus Mucor, genus Fusarium, genus Aspergillus, genus Rhodotorula, genus Entomophthora, genus Echinosporangium, and genus Saprolegnia. In another embodiment, ARA is obtained from microbial cells from the genus Mortierella, which includes, but is not limited to, Mortierella elongata, Mortierella exigua, Mortierella hygrophila, Mortierella alpina, Mortierella schmuckeri, and Mortierella minutissima.

In an even further embodiment, the microbial cells are from microalgae of the order Thraustochytriales, which includes, but is not limited to, the genera Thraustochytrium (species include arudimentale, aureum, benthicola, globosum, kinnei, motivum, multirudimentale, pachydermum, proliferum, roseum, striatum); the genera Schizochytrium (species include aggregatum, limnaceum, mangrovei, minutum, octosporum); the genera Ulkenia (species include amoeboidea, kerguelensis, minuta, profunda, radiate, sailens, sarkariana, schizochytrops, visurgensis, yorkensis); the genera Aurantiacochytrium; the genera Oblongichytrium; the genera Sicyoidochytium; the genera Parientichytrium; the genera Botryochytrium; and combinations thereof. Species described within Ulkenia will be considered to be members of the genus Schizochytrium. In another embodiment, the microbial cells are from the order Thraustochytriales. In yet another embodiment, the microbial cells are from Thraustochytrium. In still a further embodiment, the microbial cells are from Schizochytrium. In a still further embodiment, the microbial cells are chosen from genus Thraustochytrium, Schizochytrium, or mixtures thereof.

In one embodiment, the process comprises lysing microbial cells comprising a microbial oil to form a lysed cell composition. The terms “lyse” and “lysing” refer to a process whereby the wall and/or membrane of the microbial cell is ruptured. In one embodiment, the microbial cell is lysed by being subjected to at least one treatment selected from mechanical, chemical, enzymatic, physical, and combinations thereof. In another embodiment, the process comprises lysing the microbial cells comprising the microbial oil to form a lysed cell composition, wherein the lysing is selected from mechanical, chemical, enzymatic, physical, and combinations thereof.

As used herein, a “lysed cell composition” refers to a composition comprising one or more lysed cells, including cell debris and other contents of the cell, in combination with microbial oil (from the lysed cells), and optionally, a fermentation broth that contains liquid (e.g., water), nutrients, and microbial cells. In some embodiments, a microbial cell is contained in a fermentation broth or media comprising water. In some embodiments, a lysed cell composition refers to a composition comprising one or more lysed cells, cell debris, microbial oil, the natural contents of the cell, and aqueous components from a fermentation broth. In one embodiment, the lysed cell composition comprises liquid, cell debris, and microbial oil. In some embodiments, a lysed cell composition is in the form of an oil-in-water emulsion comprising a mixture of a continuous aqueous phase and a dispersed oil phase.

In general, the processes described herein can be applied to any lipid-containing microbial cells where emulsion may be formed during the process of lipids extraction. In one embodiment, the microbial cells are selected from algae, fungi, protists, bacteria, microalgae, and mixtures thereof. In another embodiment, the microalgae are selected from the phylus Stramenopiles, in particular of the family of Thraustochytrids, preferably of the genus Schizochytrium. In another one embodiment, the microbial cells described herein are capable of producing at least about 10 wt. %, at least about 20 wt. %, preferably at least about 30 wt. %, more preferably at least about 40 wt. % of their biomass as lipids. In another embodiment, the polyunsaturated lipids comprise one or any combination of DHA, EPA, and ARA.

### EXAMPLES

### Example 1

In this example, the influence of glucose concentration on emulsion formation/phase separation was examined.

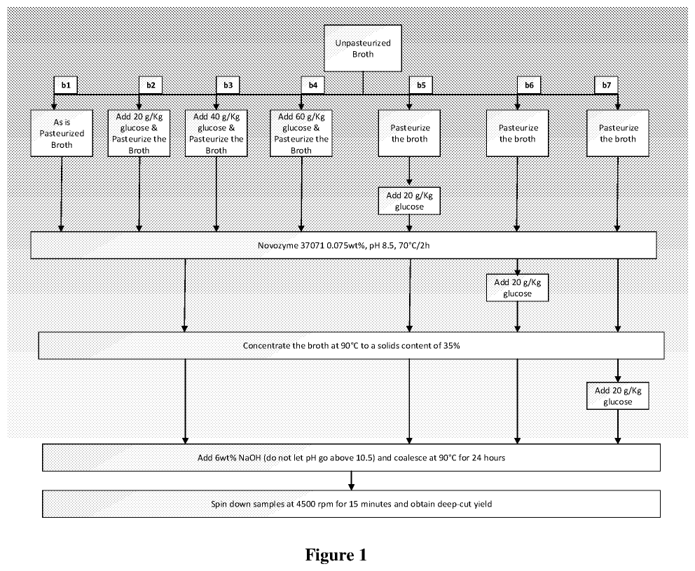

The experimental design is shown in FIG. 1. Varying amounts of glucose was added to the broth at different stages of the DSP process. Samples were withdrawn at the end of the DPS process and were analyzed for their degree of emulsion. These conditions and steps are labeled b1-b7 in FIG. 1.

b1: control, a concentration of 0.2 g/Kg residual glucose was maintained before and throughout the downstream process,

b2: 20 g/Kg glucose before pasteurization,

b3: 40 g/Kg glucose before pasteurization,

b4: 60 g/Kg glucose before pasteurization,

b5: 20 g/Kg glucose after pasteurization,

b6: 20 g/Kg glucose after cell lysis, and

b7: 20 g/Kg glucose after concentration.

### Control Experiment

In this experiment, a concentration of 0.2 g/Kg residual glucose was maintained before and throughout the downstream process, as shown in FIG. 1, b1. The degree of demulsification was measured at the end of the downstream process, by pipetting off the free oil separated after centrifugation of demulsified broth.

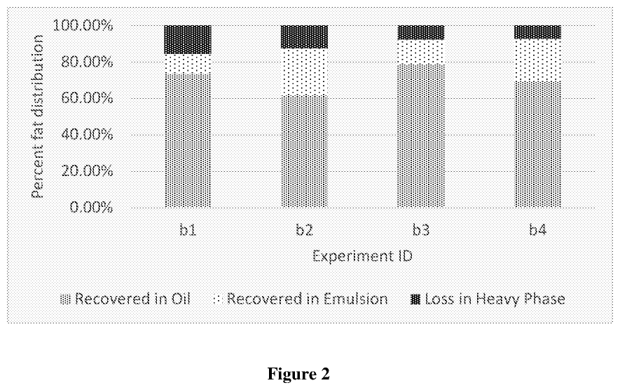

An unwashed cell broth containing microbial cells (Schizochytrium sp.) at a biomass density of over 100 g/Kg was heated to 70° C. in an agitated 3-neck round bottomed flask. After heating up the suspension, the pH was adjusted to 8.5 by using caustic soda (20 wt.-% NaOH solution), before a protease enzyme (Novozymes product 37071) was added in liquid form in an amount of 0.075 wt.-% (by weight broth). Stirring was continued for 2 hours at 70° C. After that, the lysed cell mixture was heated to a temperature of 90° C. The mixture was concentrated by evaporation of water from the lysed broth, until a total dry matter content of about 34.8 wt.-% was reached. The concentrated broth was then demulsified by changing the pH to 10.5 by addition of caustic soda (20 wt.-% NaOH solution). The total amount of caustic soda was about 6.7 wt.-% (based on the amount of initial broth weight) added in the beginning of the demulsification making sure the pH was always below 10.5. After 24 hours, the demulsified broth was neutralized to pH 7.5 by addition of sulfuric acid solution (3N). After neutralization, about 250 g of the homogenized broth sample was taken out in 50 mL centrifugation tubes and separation of the cell debris was carried out by centrifugation at 4500 rpm for 15 min. The percentage fat distributions of the oils which were recovered from the oil phase, recovered from emulsion phase, and lost in the heavy phase was measured, and was shown in FIG. 2, b1.

### Glucose Spiking Experiments

### Test 1A. Glucose Spiking Before Pasteurization

In this experiment, the influence of different concentration of residual glucose (glucose remained unconsumed in the broth after the fermentation run is complete) on demulsification was examined. Measured quantities of glucose were added to the original unpasteurized broth to make mock broths with 20 g/Kg, 40 g/Kg, and 60 g/Kg of final glucose concentration. The effect of the residual glucose on demulsification was measured by the degree of separation of the oil from the cell debris.

Unpasteurized broth, with 0.2 g/Kg residual glucose after fermentation was spiked with 20, 40 and 60 g/Kg glucose. This broth, after glucose spiking, was pasteurized at 60° C. for 1 hour in an agitated 3-neck round bottomed flask. The pasteurized broth was heated to 70° C., the pH was adjusted to 8.5 by using caustic soda (20 wt.-% NaOH solution), before a protease enzyme (Novozymes product 37071) was added in liquid form in an amount of 0.075 wt.-% (by weight broth). Stirring was continued for 2 hours at 70° C. After that, the lysed cell mixture was heated to a temperature of 90° C. The mixture was concentrated by evaporation of water from the lysed broth, until a total dry matter content of about 35 wt.-% was reached. The concentrated broth was then demulsified by changing the pH to 10.5 by addition of caustic soda (20 wt.-% NaOH solution). The total amount of caustic soda was about 6-7 wt.-% (based on the amount of initial broth weight) added in the beginning of the demulsification making sure the pH was always below 10.5. After 24 hours, the demulsified broth was neutralized to pH 7.5 by addition of sulfuric acid solution (3N). After neutralization, about 250 g of the homogenized broth sample was taken out in 50 mL centrifugation tubes and separation of the cell debris was carried out by centrifugation at 4500 rpm for 15 min. The percentage fat distributions of the oils which were recovered from the oil phase, recovered from the emulsion phase, and lost in the heavy phase was measured, and was shown in FIG. 2, b2, b3, and b4, respectively.

Test 1B Influence of 20 g/Kg Glucose on DSP when Added after Pasteurization

In this experiment, the influence of 20 g/Kg residual glucose on demulsification when added after the pasteurization step was examined. Measured quantities of glucose were added to the broth after the broth is pasteurized to make mock broths with 20 g/Kg of final glucose concentration. The effect of these residual glucose on demulsification was measured by the degree of separation of the oil from the cell debris.

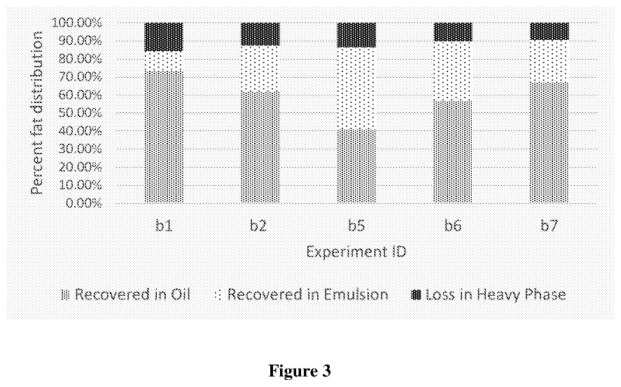

The broth (20 g/Kg glucose concentration) was heated to 70° C. in an agitated 3-neck round bottomed flask. After heating up the suspension, the pH was adjusted to 8.5 by using caustic soda (20 wt.-% NaOH solution), before a protease enzyme (Novozymes product 37071) was added in liquid form in an amount of 0.075 wt.-% (by weight broth). Stirring was continued for 2 hours at 70° C. After that, the lysed cell mixture was heated to a temperature of 90° C. The mixture was concentrated by evaporation of water from the lysed broth, until a total dry matter content of about 36.9 wt.-% was reached. The concentrated broth was then demulsified by changing the pH to 10.5 by addition of caustic soda (20 wt.-% NaOH solution). The total amount of caustic soda was about 6.5 wt.-% (based on the amount of initial broth weight) added in the beginning of the demulsification making sure the pH was always below 10.5. After 24 hours, the demulsified broth was neutralized to pH 7.5 by addition of sulfuric acid solution (3N). After neutralization, about 250 g of the homogenized broth sample was taken out in 50 mL centrifugation tubes and separation of the cell debris was carried out by centrifugation at 4500 rpm for 15 min. The percentage fat distributions of the oils which were recovered from the oil phase, recovered from the emulsion phase, and lost in the heavy phase was measured, and was shown in FIG. 3, b5.

Test 1C Influence of 20 g/Kg Glucose on DSP when Added after Cell Lysis

In this experiment, the influence of 20 g/Kg residual glucose on demulsification when added after cell lysis was examined. Measured quantities of glucose were added to the broth after the broth is lysed to make mock broths with 20 g/Kg of final glucose concentration. The effect of these residual glucose on demulsification was measured by the degree of separation of the oil from the cell debris.

Pasteurized broth, with 0.2 g/Kg residual glucose after fermentation was heated to 70° C. in an agitated 3-neck round bottomed flask. After heating up the suspension, the pH was adjusted to 8.5 by using caustic soda (20 wt.-% NaOH solution), before a protease enzyme (Novozymes product 37071) was added in liquid form in an amount of 0.075 wt.-% (by weight broth). Stirring was continued for 2 hours at 70° C. This lysed broth, with 0.2 g/Kg residual glucose after fermentation, was spiked with measured quantities of glucose to make mock broth with 20 g/Kg of final glucose concentration. After that, the lysed cell mixture was heated to a temperature of 90° C. The mixture was concentrated by evaporation of water from the lysed broth, until a total dry matter content of about 35.3 wt.-% was reached. The concentrated broth was then demulsified by changing the pH to 10.5 by addition of caustic soda (20 wt.-% NaOH solution). The total amount of caustic soda was about 6.6 wt.-% (based on the amount of initial broth weight) added in the beginning of the demulsification making sure the pH was always below 10.5. After 24 hours, the demulsified broth was neutralized to pH 7.5 by addition of sulfuric acid solution (3N). After neutralization, about 250 g of the homogenized broth sample was taken out in 50 mL centrifugation tubes and separation of the cell debris was carried out by centrifugation at 4500 rpm for 15 min. The percentage fat distributions of the oils which were recovered from the oil phase, recovered from the emulsion phase, and lost in the heavy phase was measured, and was shown in FIG. 3, b6.

Test 1D Influence of 20 g/Kg Glucose on DSP when Added after Broth Concentration

In this experiment, the influence of 20 g/Kg residual glucose on demulsification when added after the broth is concentrated was examined. Measured quantities of glucose were added to the broth after the broth is pasteurized to make mock broths with 20 g/Kg of final glucose concentration. The effect of these residual glucose on demulsification was measured by the degree of separation of the oil from the cell debris.

Pasteurized broth, with 0.2 g/Kg residual glucose after fermentation was heated to 70° C. in an agitated 3-neck round bottomed flask. After heating up the suspension, the pH was adjusted to 8.5 by using caustic soda (20 wt.-% NaOH solution), before a protease enzyme (Novozymes product 37071) was added in liquid form in an amount of 0.075 wt.-% (by weight broth). Stirring was continued for 2 hours at 70° C. After that, the lysed cell mixture was heated to a temperature of 90° C. The mixture was concentrated by evaporation of water from the lysed broth, until a total dry matter content of about 33.8 wt.-% was reached. This concentrated broth, with 0.2 g/Kg residual glucose after fermentation, was spiked with measured quantities of glucose to make mock broth with 20 g/Kg of final glucose concentration. The concentrated broth was then demulsified by changing the pH to 10.5 by addition of caustic soda (20 wt.-% NaOH solution). The total amount of caustic soda was about 6.4 wt.-% (based on the amount of initial broth weight) added in the beginning of the demulsification making sure the pH was always below 10.5. After 24 hours, the demulsified broth was neutralized to pH 7.5 by addition of sulfuric acid solution (3N). After neutralization, about 250 g of the homogenized broth sample was taken out in 50 mL centrifugation tubes and separation of the cell debris was carried out by centrifugation at 4500 rpm for 15 min. The percentage fat distributions of the oils which were recovered from the oil phase, recovered from the emulsion phase, and lost in the heavy phase was measured, and was shown in FIG. 3, b7.

### Example 2

In this example, the influence of glucose concentration on the amount of caustic soda used in the DSP process is examined.

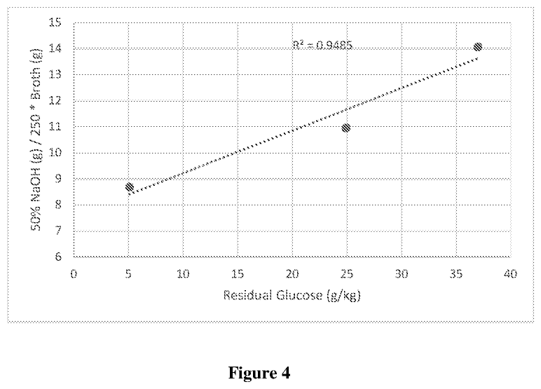

The glucose levels of a cell broth containing microbial cells (Schizochytrium sp.) at harvest were controlled down to a range between 5 and 37 g/Kg. The cell broth was heated to 60° C. in an agitated 3-neck round bottomed flask. After heating up the suspension, the pH was adjusted between 7-8 by using caustic soda (50 wt.-% NaOH solution), before a protease enzyme (Novozymes product 37071) was added in liquid form in an amount of 0.3 wt.-% (by weight broth). Stirring was continued for 2 hours at 60° C. The broth was then demulsified by maintaining the pH between 10-11 by addition of caustic soda (50 wt.-% NaOH solution) until no further drop in pH was observed. The solution was then heated to 90° C. until centrifugation at 12000 g showed visual separation of a light oil-laden phase and a heavy aqueous-laden phase. It was shown in FIG. 4 that the amount of caustic soda required for demulsification is influenced by the amount of residual glucose in the starting broth. Lower concentration of residual glucose causes less use of caustic soda.

1. A process for obtaining a microbial oil comprising one or more polyunsaturated acids from one or more microbial cells contained in a fermentation broth, wherein less than 15 g/Kg of carbohydrate is maintained in the fermentation broth during the process.

2. The process of claim 1, wherein the process further comprises:
(a) lysing the cells comprising the microbial oil to form a lysed cell composition;
(b) demulsifying the lysed cell composition to form a demulsified lysed cell composition;
(c) separating the oil from the demulsified lysed cell composition; and
(d) recovering the oil.

3. The process of claim 2, wherein 0-10 g/Kg of carbohydrate is maintained in the fermentation broth during the process.

4. The process of claim 3, wherein the said level of carbohydrate is maintained in the fermentation broth before step (a).

5. The process of claim 4, wherein the microbial cells are capable of producing at least about 10 wt. %, at least about 20 wt. %, preferably at least about 30 wt. %, more preferably at least about 40 wt. % of their biomass as lipids.

6. The process of claim 5, wherein said polyunsaturated lipids comprise one or any combination of DHA, EPA, and ARA.

7. The process of claim 6, wherein said carbohydrate is select from glucose, sucrose, dextrose, polysaccharide, and mixtures thereof.

8. The process claim 7, wherein the microbial cells are selected from algae, fungi, protists, bacteria, microalgae, and mixtures thereof.

9. The process of claim 8, wherein the microbial cells are from the genus Mortierella, genus Crypthecodinium, or order Thraustochytriales.

10. The process of claim 8, wherein the microbial cells are from the order Thraustochytriales.

11. The process of claim 10, wherein the microbial cells are from the genus Thraustochytrium, Schizochytrium, or mixtures thereof.

12. The process of claim 8, wherein the microbial cells are from Mortierella Alpina.

13. The process of any one of claims 1-4, wherein less than 18 g of caustic soda is added per 1 Kg fermentation broth at step (b).

14. An oil obtained by the process of claim 4.

15. A process for reducing the amount of caustic agent used in extracting a microbial oil comprising one or more polyunsaturated acids from one or more microbial cells contained in a fermentation broth, wherein less than 15 g/Kg of carbohydrate is maintained in the fermentation broth during the oil extraction process.

16. The process of claim 15, where less than 18 g of caustic soda is used per 1 Kg fermentation broth.

17. The process of claim 16, wherein 0-10 g/Kg of carbohydrate is maintained in the fermentation broth during the process.

18. The process of claim 17, wherein the microbial cells are capable of producing at least about 10 wt. %, at least about 20 wt. %, preferably at least about 30 wt. %, more preferably at least about 40 wt. % of their biomass as lipids.

19. The process of claim 18, wherein said polyunsaturated lipids comprise one or any combination of DHA, EPA, and ARA.

20. The process of claim 19, wherein said carbohydrate is select from glucose, sucrose, dextrose, polysaccharide, and mixtures thereof.

21. The process of claim 20, wherein the microbial cells are selected from algae, fungi, protists, bacteria, microalgae, and mixtures thereof.

22. The process of claim 20, wherein the microbial cells are from the genus Mortierella, genus Crypthecodinium, or order Thraustochytriales.

23. The process of claim 21, wherein the microbial cells are from the order Thraustochytriales.

24. The process of claim 23, wherein the microbial cells are from the genus Thraustochytrium, Schizochytrium, or mixtures thereof.

25. The process of claim 21, wherein the microbial cells are from Mortierella Alpina.

26. An oil obtained by the process of claim 17.
